(19) United States
(12) Patent Application Publication
(10) Pub. No.: US 2026/0182864 A1
(43) Pub. Date: Jul. 2, 2026

(54) METHOD AND SYSTEM FOR PSYCHOPATHOLOGICAL DIAGNOSIS USING PSYCHOPHYSIOLOGICAL STATE PARAMETERS IN VIBRATION IMAGE TECHNOLOGY

(71) Applicants: Jin Kwan CHOI, Gyeonggi-do (KR); Min Sung CHOI, Gyeonggi-do (KR)

(72) Inventor: Jin Kwan CHOI, Gyeonggi-do (KR)

(73) Assignees: Jin Kwan CHOI, Gyeonggi-do (KR); Min Sung CHOI, Gyeonggi-do (KR)

(21) Appl. No.: 19/432,198

(22) Filed: Dec. 24, 2025

(30) Foreign Application Priority Data
Dec. 27, 2024 (KR) 10-2024-0199287

(51) Int. Cl. A61B 5/11; A61B 5/00; A61B 5/16; G06V 10/50; G06V 10/62; G06V 20/52; G06V 40/20

(52) U.S. Cl. CPC A61B 5/1128; A61B 5/0077; A61B 5/1101; A61B 5/165; A61B 5/4866; A61B 5/7278; G06V 10/50; G06V 10/62; G06V 20/52; G06V 40/20

(57) ABSTRACT

A method and system for psychopathological diagnosis are proposed. The method may include capturing video of a subject, extracting a vibration image of the subject from the video, extracting, from the vibration image, the psychophysiological information, changes of energy metabolism, and a plurality of parameters related to emotional states of the subject, and performing psychopathological evaluation and diagnosis by using the changes of the psychophysiological information, the changes of the energy metabolism, and the parameters.

### CROSS-REFERENCE TO RELATED APPLICATION

This application is based on and claims priority under 35 U.S.C. § 119 to Korean Patent Application No. 10-2024-0199287, filed on Dec. 27, 2024, in the Korean Intellectual Property Office, the disclosure of which is incorporated by reference herein in its entirety.

### BACKGROUND

### Technical Field

The disclosure relates to a method and system for psychopathological diagnosis, and more particularly, to a non-contact method and system for psychopathological diagnosis based on psychophysiological information and energy metabolism derived from vibration image technology and based on a plurality of psychophysiological state parameters.

### Description of Related Art

Modern concepts of cognitive psychology are generally associated with the concepts and definitions of signal information and transmission theory and provide psychophysiological information through mathematical parameters established in information theory.

Based on statistical parameters used in information theory, long-term studies and observations have been conducted on micromovements of the human head. It has been revealed that statistically reliable correlations exist between human psychophysiological states and parameters of head micromovement information.

An interrelation between psychophysiological information (PI) and energy metabolism (EM) may be explained as a correlation between specific energy consumption in typical psychophysiological states and psychophysiological energy individually required to maintain such states. Psychophysiological energy is formed as a result of conscious or unconscious processes necessary for realizing psychophysiological processes.

An aggressive state may appear differently among individuals even in the same aggressive situation. For example, even if two people feel aggression in the same situation, their responses may differ, and such differences are influenced by individual characteristics such as age, gender, and educational level.

However, from a psychophysiological perspective, individual differences in aggression are not directly related to the amount or location of energy released in the body. Such differences appear as visible emotional signs, such as facial flushing, frequent sighing, rapid heartbeat, or specific micromovements.

The main cause of emotions being expressed outwardly lies in internal release of energy that changes psychophysiological energy within the human body. An important point here is that, as modern technology advances, natural physiological processes in the human body and chemical energy have been well understood. The manifestation of emotions may depend on progression speeds of physiological processes and on interruption and triggering processes for human thinking and movement.

Vestibular system signals are transmitted from all parts of the human body through nerve axons or wires, and changes occurring in the body are also detected, and thus the vestibular system is largely associated with the nervous system and energy regulation. Accordingly, certain emotions or stimuli may change energy regulation and energy balance in a person and may cause some changes in all physiological systems.

All psychophysiological measuring instruments indicate changes in physiological states, and when a controlled physiological system is sensitive to such changes, the instruments may function better and may acquire physiological changes more accurately.

Although the vestibular system is very sensitive to certain changes in the human body, traditional physiological signal measuring devices neither detect nor record such vestibular changes based on emotions and energy. Therefore, research on methods and systems for detecting vestibular changes is desirable.

### SUMMARY

Provided are a method and system for diagnosing a subject's psychopathological state in a non-contact manner using video.

Provided are a method and system for diagnosing a subject's psychopathological state by using a frequency histogram of a vibration image.

Additional aspects will be set forth in part in the description which follows and, in part, will be apparent from the description, or may be learned by practice of the presented embodiments of the disclosure.

According to an aspect of the disclosure, a method of diagnosing a psychopathological state includes
capturing, by using a video camera, video of a subject,
extracting, by using an image processing unit, a vibration image of the subject from the video,
extracting, by using an information extracting unit, changes of psychophysiological information and changes of energy metabolism, and a plurality of parameters related to emotional states of the subject from the vibration image, and
performing, by using a psychopathological diagnosis unit, psychopathological evaluation and diagnosis based on the changes of the psychophysiological information, the changes of the energy metabolism, and the plurality of parameters.

According to one or more embodiments,
the vibration image may include at least one of a vibration frequency, an amplitude, and a phase according to locations in each part of the subject.

According to one or more embodiments,
the extracting of the changes of the psychophysiological information, the changes of the energy metabolism, and the plurality of parameters may include extracting, by extracting frequency histograms from the vibration image, the psychophysiological information and the changes of the energy metabolism according to temporal variations.

According to one or more embodiments,
wherein the psychophysiological information at a time “t” may be calculated, by using an average value of the frequency histograms, a standard deviation, and a maximum frequency of the frequency histograms, according to Equation 1.

$$PI ( t ) = ( fps ( t ) - k {( t )}^{*} S ( t ) ) / fps ( t ) \qquad < Equation 1 >$$

wherein,
PI(t): psychophysiological information
fps(t): frames per second
k=5*sqrt (N0/100): fps-proportional constant
S(t): standard deviation of frequency histogram.

According to one or more embodiments,
the energy metabolism at a time “t” may be calculated according to Equation 2.

$$EM ( t ) = f Max ( t ) / fps ( t ) \qquad < Equation 2 >$$

wherein,
EM(t): energy metabolism
fps(t): frames per second
fMax(t): fps value at which maximum frequency of histogram appears.

According to one or more embodiments, wherein the plurality of parameters include aggression, stress, tension, balance, charisma, energy, inhibition, and neuroticism, and at least two thereof are used for evaluating and diagnosing a psychopathological state.

According to one or more embodiments, the extracting of the changes in the psychophysiological information, the changes in the energy metabolism, and the plurality of parameters may include extracting, by extracting frequency histograms from the vibration image, the changes of the psychophysiological information and the changes of the energy metabolism according to temporal variations, and
calculate the psychophysiological information and the energy metabolism, both at a time “t”, by using an average value of the frequency histograms, a standard deviation, and a maximum frequency of the frequency histograms, according to Equations 3 and 4.

$$PI ( t ) = ( fps ( t ) - k {( t )}^{*} S ( t ) ) / fps ( t ) \qquad < Equation 3 >$$

wherein,
PI(t): psychophysiological information
fps(t): frames per second
k=5*sqrt (N0/100): fps-proportional constant
S(t): standard deviation of frequency histogram.

$$EM ( t ) = f Max ( t ) / fps ( t ) \qquad < Equation 4 >$$

wherein,
EM(t): energy metabolism
fps(t): frames per second
fMax(t): fps value at which maximum frequency of histogram appears.

According to another aspect of the disclosure, a system for diagnosing a psychopathological state includes
a video camera configured to capture a video from a subject,
an image processing unit configured to extract a vibration image of the subject from the video,
an information extracting unit configured to extract, from the vibration image, the changes in the psychophysiological information and changes in energy metabolism of the subject and a plurality of parameters related to emotional states of the subject, and
a psychopathological diagnosis unit configured to perform psychopathological evaluation and diagnosis based on the changes of the psychophysiological information, the changes of the energy metabolism, and the plurality of parameters.

According to one or more embodiments,
the information extracting unit may be further configured to extract, by extracting frequency histograms from the vibration image, the changes of the psychophysiological information and the changes of the energy metabolism according to temporal variations.

According to one or more embodiments,
the information extracting unit may be further configured to calculate the psychophysiological information at a time “t”, by using an average value of the frequency histograms, a standard deviation, and a maximum frequency of the frequency histograms, according to Equation 5.

$$PI ( t ) = ( fps ( t ) - k {( t )}^{*} S ( t ) ) / fps ( t ) \qquad < Equation 5 >$$

wherein,
PI(t): psychophysiological information
fps(t): frames per second
k=5*sqrt (N0/100): fps-proportional constant
   S(t): standard deviation of frequency histogram.

According to one or more embodiments, the energy metabolism at a time “t” is calculated according to Equation 6.

The actual psychophysiological information (PI) has a value obtained by multiplying the variation ratio by 100, and therefore has a range of values from 0 to 100. Further, because this may be inversely proportional to a standard deviation S value of frequency histograms, when the standard deviation S value decreases, the psychophysiological information PI conversely increases, and when the standard deviation S value increases, the psychophysiological information PI conversely decreases.

Accordingly, the psychophysiological information PI indicates a degree of stability/instability of psychophysiology.

For example, a large value of the psychophysiological information PI means that the standard deviation S value of the frequency histogram is small and that psychophysiological variability is almost absent and is maintained at a constant level. Ultimately, this means that the psychophysiology may be in a stable state.

Conversely, a small value of psychophysiological information PI means that a standard deviation S value of the frequency histograms is large and that the psychophysiological variability changes very greatly. Ultimately, this means that the psychophysiology may be in an unstable state.

$$EM ( t ) = f Max ( t ) / fps ( t ) \qquad < Equation 6 >$$

wherein,
EM(t): energy metabolism
fps(t): frames per second
fMax(t): fps value at which maximum frequency of histogram appears.

In practice, a value of energy metabolism EM may use a value obtained by multiplying a ratio by 10, and therefore may have a range from 0 to 10. Further, because this may be proportional to an fMax value that is a value of fps at which a maximum frequency of a FH appears, when the fMax value decreases, a value of the energy metabolism EM also decreases, and when the fMax value increases, a value of the energy metabolism EM also increases. Accordingly, the energy metabolism EM indicates a degree of maximum vitality.

According to one or more embodiments, wherein the information extracting unit is further configured to extract at least two of the plurality of parameters related to emotional states, including aggression, stress, tension, balance, charisma, energy, inhibition, and neuroticism.

According to one or more embodiments, the information extracting unit may be further configured to:
extract, by extracting frequency histograms from the vibration image, the psychophysiological information and the changes of the energy metabolism according to temporal variations; and
calculate the psychophysiological information and the energy metabolism, both at a time “t”, by using an average value of the frequency histograms, a standard deviation, and a maximum frequency of the frequency histograms, according to Equations 7 and 8.

$$PI ( t ) = ( fps ( t ) - k {( t )}^{*} S ( t ) ) / fps ( t ) \qquad < Equation 7 >$$

wherein,
PI(t): psychophysiological information
fps(t): frames per second
k=5*sqrt (N0/100): fps-proportional constant
S(t): standard deviation of histogram.

$$EM ( t ) = f Max ( t ) / fps ( t ) \qquad < Equation 8 >$$

wherein,
EM(t): energy metabolism
fps(t): frames per second
fMax(t): fps value at which maximum frequency of histogram appears.

According to one or more embodiments, wherein the information extracting unit may be further configured to extract at least two of the plurality of parameters related to emotional states, including aggression, stress, tension, balance, charisma, energy, inhibition, and neuroticism.

According to one or more embodiments, the psychopathological diagnosis unit may further perform psychopathological evaluation and diagnosis for the subject by applying an artificial intelligence model trained with the changes of the psychophysiological information, the changes of the energy metabolism, and at least two of the plurality of parameters selected from aggression, stress, tension, balance, charisma, energy, inhibition, and neuroticism.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates a graph defining a psychological state according to variations of psychophysiological information (PI) and energy metabolism (EM) as parameters applied in one or more embodiments;

FIG. 2 illustrates PI-EM variation graphs obtained according to an embodiment, wherein a graph (A) is a graph of a depressed patient and a graph (B) is a graph of a healthy person for comparison;

FIG. 3 is a graph of time-series data obtained according to an embodiment, showing a relationship between PI and EM in a diagnostic case of a depressed patient with very low PI;

FIG. 4 is a graph showing a relationship between PI and EM as time-series data, obtained from a diagnostic case of an anxiety-disorder patient according to an embodiment;

FIG. 5 illustrates, as a graph obtained according to a method of an embodiment, a diagnostic case of a burn-out state in which variations in EM are very low;

FIG. 6 illustrates a frequency histogram measured according to a method of an embodiment, in which a frequency domain representing a burn-out state appears;

FIG. 7 illustrates a vibration image representing emission of bio-energy around a subject's body, the vibration image extracted according to an embodiment from an image of the subject's body;

FIG. 8 illustrates a normal distribution of a frequency histogram generated according to an embodiment;

FIG. 9 illustrates a low-frequency histogram (A) and a high-frequency histogram (B) generated according to an embodiment;

FIG. 10 illustrates frequency histograms of a worst case and a best case, wherein a frequency histogram (A) is a frequency histogram of a subject having psychopathological issues, while a frequency histogram (B) is a frequency histogram of a subject in a healthy and good state;

FIG. 11 illustrates a frequency histogram of a healthy and good normal state; and

FIG. 12 illustrates a block diagram showing a schematic configuration of a system for evaluating and diagnosing a psychopathological state according to an embodiment.

### DESCRIPTION OF THE EMBODIMENTS

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, preferred embodiments of the present inventive concept are described in detail with reference to the accompanying drawings. However, the embodiments of the present inventive concept may be modified into various other forms, and the scope of the present inventive concept shall not be construed as being limited to the embodiments described below in detail. It is desirable that the embodiments of the present inventive concept be interpreted as being provided to more fully explain the present inventive concept to one of ordinary skill in the art. The same signs denote the same elements throughout. Furthermore, various elements and areas in the drawings are schematically drawn. Therefore, the present inventive concept is not limited by the relative sizes or distances illustrated in the accompanying drawings.

Terms such as first, second, etc. may be used to describe various components, but the components are not defined by these terms. The terms are used only for distinguishing one element from another element. For example, without deviating from the scope of the claims of the present inventive concept, a first element may be referred to as a second element, and vice versa.

The terms used in the present specification are used only for describing particular embodiments and are not intended to limit the present inventive concept. A singular expression may include a plural expression, unless an apparently different meaning is indicated in the context. With respect to the present application, it will be further understood that the expressions “comprises” and “comprising” used herein specify the presence of stated features, integers, steps, operations, members, components, and/or groups thereof, but do not preclude the presence or addition of one or more other features, integers, operations, members, components, and/or groups thereof.

Unless otherwise defined, all of the terms used herein including technical terms and scientific terms have the same meaning as the meaning commonly understood by one of ordinary skill in the art. Also, the terms commonly used and having the meanings as defined in dictionaries shall be understood to have consistent meanings with the corresponding terms in the context of relevant arts, and unless explicitly defined so herein, the meanings of the terms shall not be understood to be excessively formal.

In cases where an embodiment may be implemented differently, the specific process steps may be performed in an order different from the described sequence. For example, two consecutively described processes may be performed substantially simultaneously or may be performed in a reverse order to the described sequence.

Because the human vestibular organ mentioned in the disclosure is connected with sensory organs, neural organs, and other parts of the human body, three-dimensional trajectories of head movements are used in one or more embodiments as micromovements as a source for evaluating a psychopathological state.

Measuring psychophysiological information (PI) and energy metabolism (EM) in the view of a person's energy consumption is simple and definite as a method that organizes bases and concepts of modern science. The issue of defining emotional-state information for a person is never simple. At the same time, every adult may easily say, at a level that they perceive, how well they feel at that moment and how happy they are at that moment.

A frequency histogram (FH) of a psycho-physiological state (PPS) represents an overall distribution of micromovements of the head during a measuring time.

A histogram similar to a Gaussian distribution of a general rule in a distribution map of the FH indicates a normal state. An asymmetric FH having a plurality of peaks indicates an abnormal state in which pathological phenomena exist when compared with a normal state, and this abnormal state has unique characteristics.

A vibration image may represent vibration distributions in space and time. The FH represents temporal vibration distributions regardless of partial movements of action units (AU) defined on the face, and differs from the facial action coding system (FACS).

The FACS encodes simple emotional facial expressions as internal facial-control responses without including responses of head movements, whereas the FH of a vibration image provides basic information for calculating the parameters related to emotional states as temporal vibration distributions.

The FH enables use of concepts and principles of information theory, statistics, thermodynamics such as energy and information, a probability density function, and entropy for emotional theory and operations.

A relationship between such psychophysiological information PI and energy metabolism EM may be represented on x-y orthogonal coordinates. Displaying a psychophysiological emotional state as a percentage is simplest, and therefore the psychophysiological information PI for a person's emotional state may be represented as a percentage (%) on the y-axis of orthogonal coordinates in vibration image technology. In practice, the psychophysiological information PI, as time-series data, may be represented in a form of a straight line or a curve connecting points corresponding to individual pieces of information.

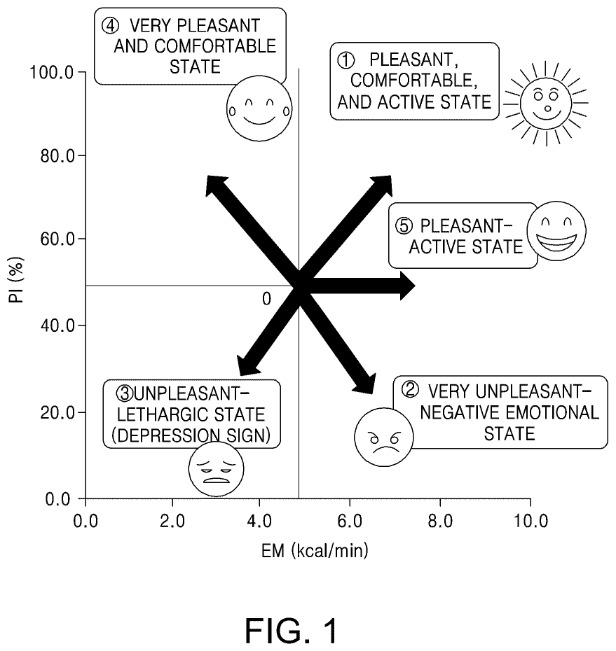

FIG. 1 illustrates a graph defining psychological states according to variations of the psychophysiological information PI and the energy metabolism EM.

A vertical axis (a Y-axis) represents the psychophysiological information PI, indicates positive or negative psychological aspects of a state, and displays values from 0 to 100%. A horizontal axis (an X-axis) represents the energy metabolism EM, indicates an amount of energy consumption according to an activity level, and has a unit of Kcal/min.

### (1) a Pleasant, Comfortable, and Active State

This state is a state in which PI and EM are both high, where a person moves actively and feels positive and pleasant emotions.

### (2) Very Unpleasant-Negative Emotional State

This state is a state in which PI is low and EM is high, meaning that the energy consumption is high, that the psychological state is negative, and that emotions such as anger and impulsiveness are felt.

### (3) Unpleasant-Lethargic State (Depression Sign)

This state is a state in which PI and EM are both low, where the energy consumption is low and the psychological state is negative, representing depression or lethargy.

### (4) Very Pleasant and Comfortable State

This state, as a state in which PI is high but EM is low, although activity level is low, represents a comfortable and positive state.

### (5) Pleasant-Active State

This state, where PI and EM levels are neutral and both PI and EM are balanced at appropriate levels, generally means a pleasant and active state.

A 100% level of PI represents a happy state or a Nirvana state, and a level 0 corresponds to a state in which information exchange between physiological systems of a person stops, for example, a state of death, and thus a zero PI level is excluded from diagnosis in determining psychophysiological states according to the disclosure.

The following describes a relationship between PI and EM for a depressed patient. Data presented below as samples are result data of testing a method of the disclosure.

A total of 748 soldiers participated as subjects in the experiment. For these subjects, both a military service adaptation test and a PPS test using a vibration image by a system according to the disclosure were performed. The age distribution of the subjects was 20 to 40 years, with 738 males and 10 females.

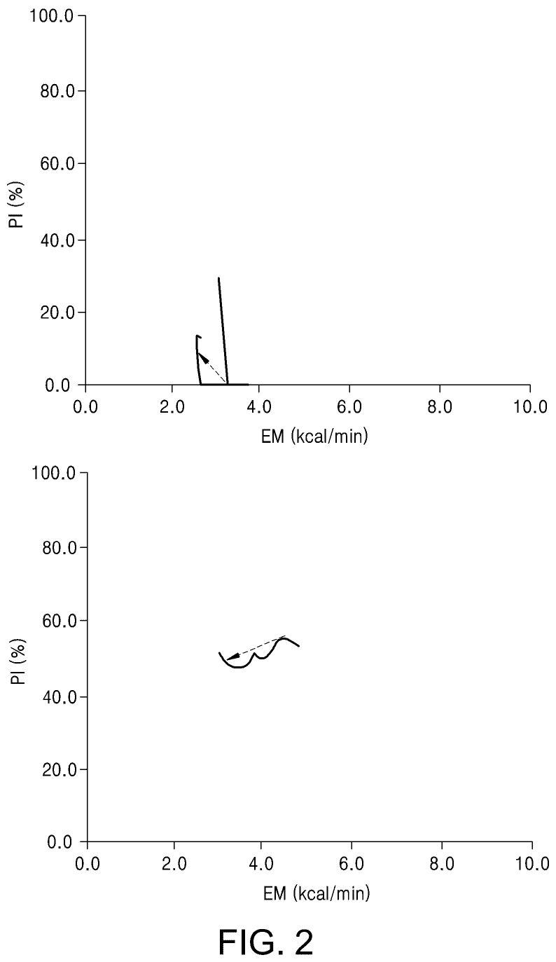

FIGS. 2 (A) and (B) are graphs of PI-EM variations of a depressed patient and a healthy person for comparison, obtained according to one embodiment.

As can be seen by comparing FIGS. 2(A) and (B), a subject having depression has a low level of PI compared to a healthy subject, whereas a joyful and pleasant healthy person has a high PI level.

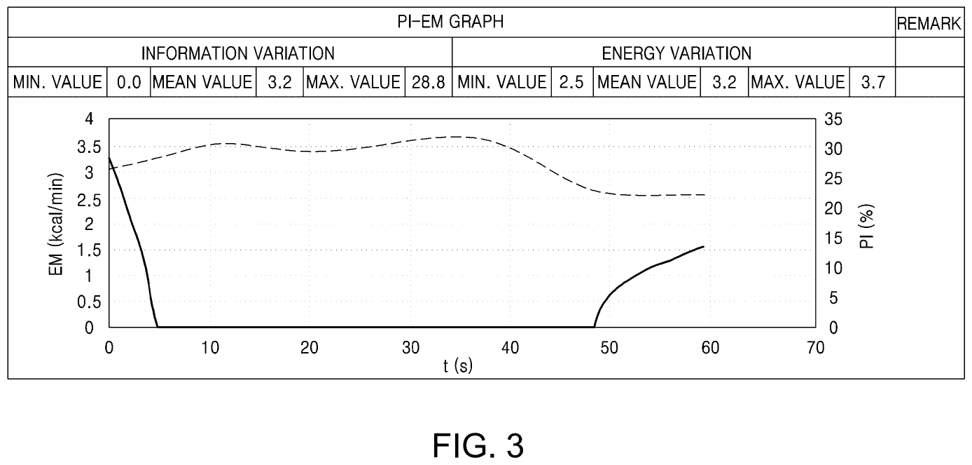

FIG. 3 is a graph of time-series data obtained according to an embodiment, showing a relationship between PI and EM in a diagnostic case of a depressed patient with a very low PI.

The PI indicated by a solid line decreases until about 5 seconds after the test starts, deteriorates to a zero state until about 48 seconds, and slightly increase s after about 48 seconds, with a minimum value of zero (0), a maximum value of 28.8, and an average value of 3.2, indicating a very low state. For a healthy person, a PI level ranges from about 40 to 60.

The EM indicated by a dotted line slightly increases immediately after the test and decreases from about 36 seconds. This case is about a patient diagnosed with very severe depression.

In addition, results of statistical analysis on depression diagnosis using other parameters are as follows.

Table 1 below shows average values M of parameters measured from a normal group and a depressed patient group.

TABLE 1

| | NORMAL | DEPRESSION | | |
|---|---|---|---|---|
| ITEM | GROUP | GROUP | t | p-value |
| Aggression (M1) | 42.01 | 42.32 | −0.3284 | 0.7427 |
| Stress (M2) | 31.90 | 30.48 | 1.9371 | 0.0431* |
| Tension (M3) | 28.27 | 27.01 | 1.6170 | 0.1598 |
| Suspect (M4) | 33.86 | 33.36 | 1.0775 | 0.2816 |
| Balance (M5) | 58.66 | 59.15 | −0.514 | 0.6074 |
| Charisma (M6) | 85.44 | 65.92 | −0.3344 | 0.7382 |
| Energy (M7) | 19.35 | 21.51 | −2.7206 | 0.0048** |
| Self-regulation (M8) | 61.82 | 62.29 | −0.508 | 0.61186 |
| Inhibition (M9) | 17.44 | 17.83 | −1.0603 | 0.2917 |
| Neuroticism (M10) | 29.24 | 28.1 | 1.0010 | 0.3171 |

Table 2 below shows variability V1-V10 of parameters measured from the normal group and the depressed patient group.

TABLE 2

| | NORMAL | DEPRESSION | | |
|---|---|---|---|---|
| ITEM | GROUP | GROUP | t | p-value |
| Aggression (V1) | 14.78 | 14.11 | 1.0789 | 0.281 |
| Stress (V2) | 15.16 | 12.99 | 2.538 | 0.0113* |
| Tension (V3) | 26.62 | 28.76 | −1.8094 | 0.0708 |
| Suspect (V4) | 10.79 | 9.77 | 2.3042 | 0.0215* |
| Balance (V5) | 13.70 | 13.34 | 0.4453 | 0.6562 |
| Charisma (V6) | 15.61 | 12.35 | 1.0027 | 0.3163 |
| Energy (V7) | 22.49 | 18.78 | 2.828 | 0.0045** |
| Self-regulation (V8) | 10.72 | 9.57 | 1.7285 | 0.0943 |
| Inhibition (V9) | 16.67 | 15.56 | 2.0533 | 0.0404 |
| Neuroticism (V10) | 36.37 | 25.20 | 1.5567 | 0.1199 |

Table 3 below shows indicators of parameters measured from the normal group and the depression group.

TABLE 3

| | NORMAL | DEPRESSION | | |
|---|---|---|---|---|
| ITEM | GROUP | GROUP | t | p-value |
| Brain fatigue degree | −1.07 | −0.89 | −1.0574 | 0.2907 |
| Concentration degree | 40.77 | 43.64 | −1.8010 | 0.0721 |
| Pleasure/Displeasure | 43.34 | 49.95 | −2.6110 | 0.0092** |
| Emotional variability | 18.25 | 17.04 | 2.1775 | 0.0297* |
| EM-Min. value | 2.62 | 2.72 | −1.3230 | 0.1780 |
| EM-Max. value | 3.90 | 4.02 | −1.0846 | 0.2809 |
| EM-Mean value | 3.17 | 3.33 | −1.7010 | 0.0922 |
| PI-Min. value | 28.24 | 31.39 | −2.5008 | 0.0096** |
| PI-Max. value | 50.61 | 51.44 | −0.7940 | 0.4274 |
| PI-Mean value | 38.51 | 40.57 | −1.8078 | 0.0490* |
| P21 Indicator | 0.02 | −0.01 | 2.5715 | 0.0098** |
| P22 Indicator | 0.01 | 0.05 | 2.6053 | 0.0083** |

Above,
*p < 0.05,
**p < 0.01

As shown in the tables above, diagnostic signs appeared in six parameter indicators at a 99% confidence level that influence depression diagnosis, including the mean value M7 and the variability V7 of energy, the pleasure/displeasure indicator, the minimum value of psychophysiological information PI, and the P21 and P22 indicators.

Further, at a 95% confidence level in depression diagnosis, diagnostic signs appeared through five parameters including a stress mean value M2, a stress variability V2, a suspect variability V4, an emotional variability, and a PI mean value.

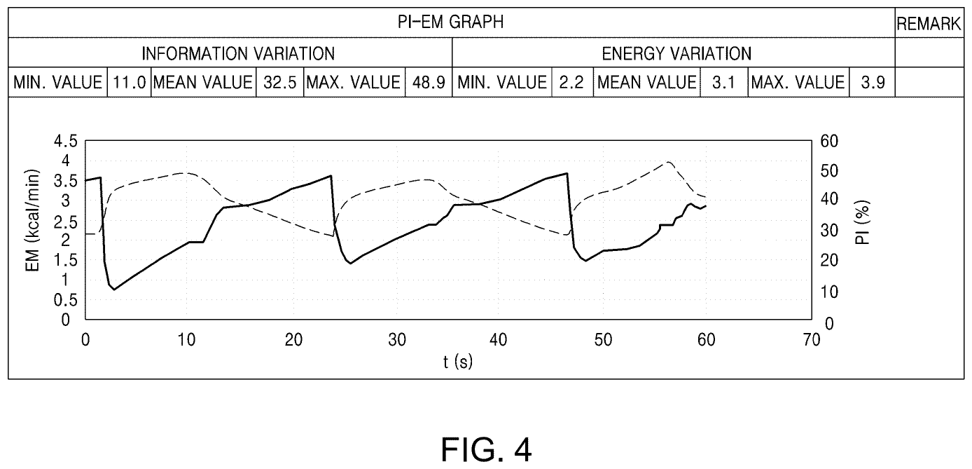

FIG. 4 illustrates, as a graph of time-series data, a PI-EM relationship of a diagnostic case of an anxiety-disorder patient measured according to a method of an embodiment, and illustrates variations in energy levels and PI.

As illustrated in FIG. 4, a graph of PI (solid line) repeats increasing and decreasing regularly, and a graph of EM (dotted line) also repeatedly increases and decreases, showing a recurring pattern of variations in both parameters.

The two parameters of PI and EM exhibit patterns in which each repeatedly increases and decreases.

Meanwhile, it is possible to psychopathologically diagnose attention deficit hyperactivity disorder (ADHD) based on relationships among a plurality of parameters.

Table 4 below shows parameters having large differences between normal children (Control, N=37) and children with attention-deficit/hyperactivity disorder (ADHD, N=48).

TABLE 4

| | Average of 10 parameters | | | | |
|---|---|---|---|---|---|
| | | Control | Average | | |
| | ADHD | group | difference | t-Test | |
| Item | (N = 48) | (N = 37) | value | t-value | p-value |
| Aggression(T1) | 54.0 | 41.8 | 12.2 | 10.062 | 0.0001** |
| Stress (T2) | 22.4 | 30.7 | 8.3 | −8.366 | 0.0001** |
| Tension (T3) | 26.3 | 25.9 | 0.4 | 0.198 | 0.844 |
| Suspect (T4) | 34.4 | 32.5 | 1.9 | 2.446 | 0.017 |
| Charm (T5) | 70.0 | 72.0 | −2.0 | −1.295 | 0.199 |
| Charisma (T6) | 79.8 | 65.1 | 4.7 | 9.144 | 0.0001** |
| Energy (T7) | 38.4 | 23.5 | 14.9 | 6.847 | 0.0001** |
| Self-regulation | 74.3 | 67.8 | 6.5 | 4.247 | 0.0001** |
| (T8) | | | | | |
| Inhibition (T9) | 19.3 | 19.4 | −0.1 | −0.065 | 0.948 |
| Neuroticism | 39.1 | 29.6 | 9.5 | 3.361 | 0.001** |
| (T10) | | | | | |

**p < 0.01: significant differences at a 99% confidence level

Among the ten parameters measured from an ADHD group (N=48) and normal children (N=37), six parameters show significant differences at a 99% confidence level, and these parameters include aggression, stress, charisma, energy, self-regulation, and neuroticism.

As mentioned above, verification of ADHD patients is facilitated by using parameters that have been highly validated for distinguishing ADHD children and normal children. By using diagnostic criteria for distinguishing normal children and ADHD children based on these verification results, diagnoses could be made with the accuracy rates shown below.

TABLE 5

to

| | ADHD | Normal | | Accuracy |
|---|---|---|---|---|
| From | children | children | Total | (%) |
| ADHD children | 47 | 1 | 48 | 97.9% |
| Normal children | 3 | 34 | 37 | 91.9% |
| Total | 50 | 35 | 85 | 94.9% |

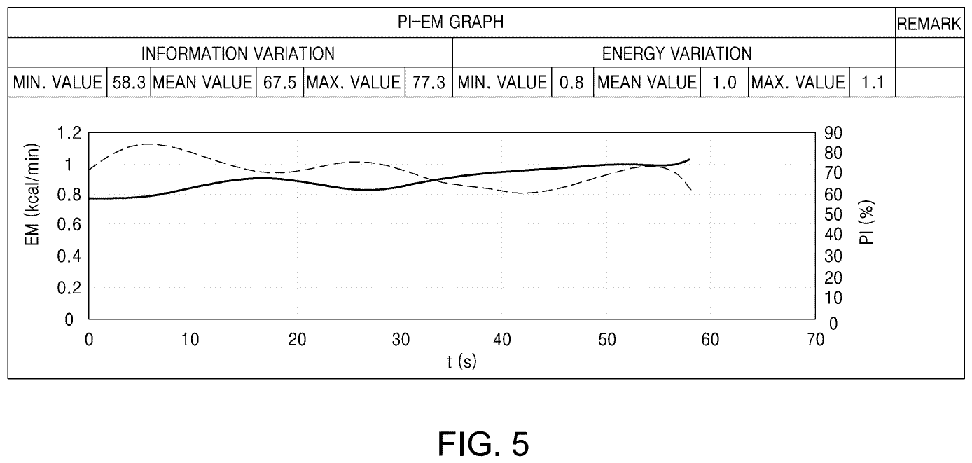

FIG. 5 illustrates variations in states of two parameters related to diagnosis of a burn-out state.

Referring to FIG. 5, a level of EM (dotted line) varying on a time axis shows very low values during a test time, with a mean value of 1.0 and ranging from a minimum value of 0.8 to a maximum value of 1.1, and a level of PI indicated by a solid line shows a slightly higher state than that of a normal person, with a mean value of 67.5 in the graph. The state of FIG. 5 represents a burn-out state in which psychological energy is very low.

This burn-out state may also be confirmed below through Table 6, in which indicators related to bio-vitality and energy among ten parameters appear lower than normal ranges.

TABLE 6

| | | | Min. | AVG. | Max. |
|---|---|---|---|---|---|
| ITEMS | RANGE | Decision | value | value | value |
| Active/Aggression | 20-50 | Low | 10.7 | 15.0 | 25.0 |
| Stress | 20-40 | Normal | 21.3 | 34.1 | 37.8 |
| Tension | 15-40 | Normal | 13.3 | 38.1 | 53.8 |
| Suspicion | 20-50 | Normal | 23.4 | 29.3 | 34.2 |
| Balance | 40-80 | Low | 0.0 | 32.0 | 53.6 |
| Charisma | 50-80 | Low | 19.9 | 48.3 | 85.0 |
| Energy | 20-40 | Low | −0.6 | 3.0 | 5.0 |
| Self-regulation | 50-80 | Low | 10.0 | 42.3 | 56.0 |
| Inhibition | 15-25 | Normal | 11.7 | 15.4 | 24.9 |
| Neuroticism | 20-50 | Normal | 0.2 | 23.5 | 36.4 |

In Table 6, aggression is measured as 15.0 (normal range: 20-50), energy is measured as 3.0 (normal range: 20-40), and balance is measured as 32.0 (normal range: 40-80), indicating very low values. In a FH representing bio-vitality, values are also distributed in a low-frequency range, and a mean value is 0.47, indicating a very low state.

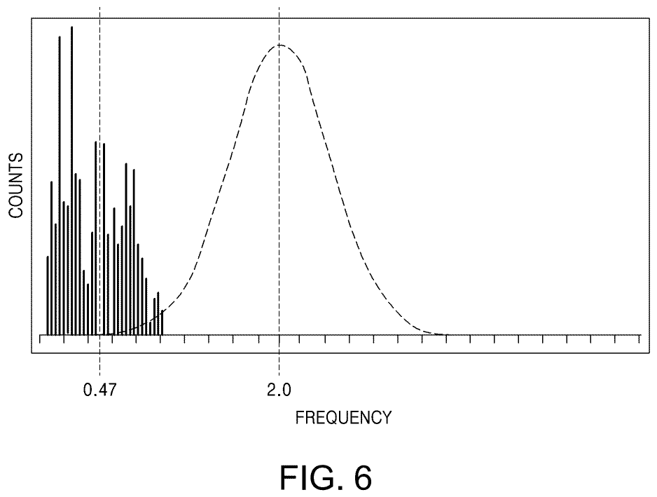

FIG. 6 illustrates a FH measured according to a method of an embodiment, in which a frequency domain representing a burn-out state appears.

As illustrated in FIG. 6, a frequency band representing a burn-out state is distributed in a low-frequency range even in the FH representing bio-vitality, and a mean value is 0.47, indicating a very low state, whereas a Gaussian-shaped histogram of 2.0 indicates a psychopathologically normal state.

As described above, when performing psychopathological evaluation and diagnosis, a psychopathological state may be comprehensively diagnosed by combining a relationship between PI and EM and the plurality of parameters.

Hereinafter, a method of determining or diagnosing psychopathological signs by using the plurality of parameters serving as grounds for such psychopathological determination is described.

A method and a system according to one or more embodiments basically detect psychophysiological bioinformation by a non-contact measuring method and use this to evaluate and diagnose psychopathological signs of a subject as described above.

Specifically, a method and a system according to one or more embodiments analyze micromovements of a human head that are directly related to vestibular-system actions causing changes in emotions and energy balance, and evaluate and diagnose a psychopathological state of a subject.

A non-contact psychopathological diagnostic system may be based on a computer, processes video obtained by a video camera to extract a vibration image, and diagnoses psychopathological signs of a subject by detecting psychophysiological-state information and energy metabolism from the vibration image, using analysis software.

Such a diagnostic system may include
a video-camera system configured to capture a subject and generate video,
an image processing unit configured to extract a vibration image of the subject from the video,
an information extracting unit configured to extract the changes of psychophysiological information and changes of energy metabolism of the subject and a plurality of parameters related to emotional states from the vibration image, and
a psychopathological diagnosis unit configured to perform psychopathological evaluation and diagnosis based on the changes of the psychophysiological information, the changes of the energy metabolism, and the plurality of parameters.

According to one or more embodiments, a display unit for visualizing an evaluated and diagnosed psychopathological state may further be included, and this may be included in the diagnosis unit.

The image processing unit, the information extracting unit, and the psychopathological diagnosis unit may be provided by hardware of a computer system and analysis software activated and executed on the system.

A method of psychopathological diagnosis may include capturing, by using a video camera, video of a subject, extracting, by using an image processing unit, a vibration image of the subject from the video, extracting, by using an information extracting unit, changes of psychophysiological information and changes of energy metabolism, and a plurality of parameters related to emotional states of the subject from the vibration image, and performing, by using a psychopathological diagnosis unit, psychopathological evaluation and diagnosis based on the changes of the psychophysiological information, the changes of the energy metabolism, and the plurality of parameters.

The vibration image is based on head movements characterized by information of a vestibuloemotional reflex (VER) and provides the plurality of parameters extracted by the information extracting unit.
the extracted plurality of parameters may include aggression, stress, tension, balance, charisma, energy, inhibition, and neuroticism, and at least two of the parameters are used for evaluating and diagnosing psychopathological states.

A vibration image extracted from video reflects head movements related to functional states of the body in real time and provides variables calculated by differentiating and integrating the image according to unit-frame analysis.

As described above, the FH of the PPS represents an overall distribution of micromovements of the head during a measurement period.

A histogram similar to a Gaussian distribution of a general rule in a distribution map of a FH indicates a normal state. An asymmetric FH having a plurality of peaks indicates an abnormal state in which pathological phenomena exist when compared with a normal state, and this abnormal state has unique characteristics.

The FH enables use of concepts and principles of information theory, statistics, thermodynamics such as energy and information, probability density functions (PDFs), and entropy for emotion-theory operation.

According to an example embodiment, characteristics of the FH depend on human emotional states and individual traits. Examples of such FHs indicate that psychophysiological information and energy-metabolism levels are mutually adjusted.

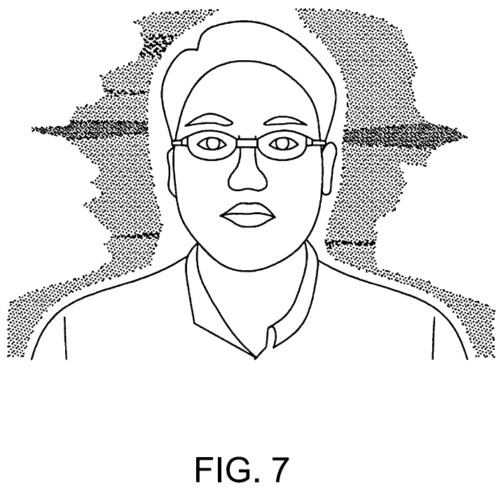

FIG. 7 illustrates a vibration image, extracted according to an embodiment, representing emission of bio-energy around a subject's body image.

As described above, a vibration image extracted from a video reflects head movements related to functional states of the body in real time and provides variables calculated by differentiating and integrating the vibration image according to unit-frame analysis. Each of the variables may be expressed in terms of an amplitude A and a frequency F.

$${A}_{x , y} = \frac{1}{N} {\sum }_{I = 1}^{N} \vert {U}_{x , y , i} - {U}_{x , y , ( i + 1 )} \vert \qquad [ Equation 1 ]$$

wherein A indicates amplitude, N indicates number of frames, U_(x,y,i) indicates signal amplitude at point x, y in i-th frame, and U_(x,y, (i+1)) indicates signal amplitude at point x, y in the i+1-th frame. The frequency components are calculated according to Equation 2.

$${F}_{x , y} = \frac{{F}_{in}}{N} {\sum }_{i = 1}^{N} \{ \begin{matrix}\vert {U}_{x , y , i} - {U}_{x , y , ( i + 1 )} \vert > 0 : 1 \\ otherwise \text{:} 0\end{matrix} \} . \qquad [ Equation 2 ]$$

In the disclosure, signal components corresponding to brainwave signals are extracted from parameters, and through this, a psychopathological diagnosis of a subject is performed.

First, extraction or calculation of parameters related to emotional states from vibration images is described in detail.

Acquisition of information on a biological subject's aggression level is performed by constructing a frequency-distribution histogram and measuring parameters related to emotional states of the subject's head according to the histogram.

### Aggression (T1)

The parameter aggression T1 indicates a maximum value of a frequency distribution and a standard deviation of facial-vibration frequencies according to a frequency bar graph. As the standard deviation becomes larger and the maximum value of the distribution becomes higher, a value of the aggression parameter increases. An aggressive person has higher frequencies of head micromovements and exhibits a wider dispersion in movements of various points of the head.

$$T 1 = \frac{Fm + 4 * \sqrt{\frac{1}{n} {\sum }_{1}^{n} {( {F}_{i} - \overline{F} )}^{2}}}{2 Fin} \qquad [ Equation 3 ]$$

wherein,
Fm: maximum frequency of frequency-distribution density in a histogram
Fi: an aggregated amount of the “I” frequency of a frequency-distribution density, acquired during
N frames in the histogram
Fin: processing frequency of a vibration image
n: an aggregated amount including inter-frame differences exceeding a threshold among N frames.

### Stress (T2)

The parameter stress level T2 is determined according to a degree of imbalance in micromovements between the right and left regions of the head. A large difference in amplitudes and frequencies of movements between the right and left regions of the head indicates a high value of the stress parameter.

$$T 2 = \frac{{\sum }_{1}^{k} ( \frac{\vert {A}_{L}^{i} - {A}_{R}^{i} \vert}{{A}_{max}^{i}} + \frac{\vert {F}_{L}^{i} - {F}_{R}^{i} \vert}{{F}_{max}^{i}} )}{2 n} \qquad [ Equation 4 ]$$

wherein,

$${A}_{L}^{i} :$$

Total amplitude of vibration images of the left region in the “I” column of the subject

$${A}_{R}^{i} :$$

Total amplitude of vibration images of the right region in the “I” column of the subject

$${A}_{max}^{i} :$$

Max. Value between

$${A}_{L}^{i} and {A}_{R}^{i}$$

$${F}_{L}^{i} :$$

Max. frequency of vibration Images of the left region in the “I” column of the subject

$${F}_{R}^{i} :$$

Max. frequency of vibration Images of the right region in the “I” column of the subject

$${F}_{max}^{i} :$$

Max. Value between

$${F}_{L}^{i} and {F}_{R}^{i}$$

n: number of columns associated with the subject.

### Tension (T3)

The parameter tension level T3 is determined according to how large a high-frequency portion of a vibration-frequency spectrum is relative to a total area of a spectrum of micromovement frequencies of the head. As a density of a high-frequency portion increases, a value of the tension parameter becomes higher.

$$T 3 = \frac{{\sum }_{\frac{{f}_{max}}{2}}^{{f}_{max}} {P}_{i} ( f )}{{\sum }_{0 , 1}^{{f}_{max}} {P}_{i} ( f )} \qquad [ Equation 5 ]$$

wherein,
Pi(f): Power spectrum of a vibration-image frequency distribution
fmax: Max. frequency of a spectrum of a vibration-image frequency distribution.

### Suspect (T4)

The parameter suspect level T4 indicates an overall negative emotional state and represents an emotional level of a potentially dangerous subject.

$$T 4 = \frac{\sum {[ y ( x ) * K - {y}^{′} ( x ) ]}^{2}}{\sum {[ {y}^{′} ( x ) ]}^{2}} \qquad [ Equation 6 ]$$

wherein,
K: Generalized correlation coefficient of an acquired frequency histogram.

$$K = \frac{\sum {y}^{′} ( x )}{\sum y ( x )}$$

wherein, y′ indicates density of a normal distribution.

$${y}^{′} = \frac{1}{\sqrt{2 π}} {e}^{- \frac{{( x - M )}^{2}}{2 {σ}^{2}}}$$

### Balance (T5)

The parameter balance T5 is determined according to a frequency bar graph and indicates a degree of similarity of a current frequency bar graph to a normal distribution. A frequency bar graph showing a high similarity to a normal distribution indicates a high level of balance. A large deviation from the normal distribution indicates a low value of the balance parameter.

$$T 5 = \frac{\sum {[ y ( x ) * K - {y}^{′} ( x ) ]}^{2}}{\sum {[ {y}^{′} ( x ) ]}^{2}} \qquad [ Equation 7 ]$$

$$K = \frac{\sum {y}^{′} ( x )}{\sum y ( x )}$$

wherein, y′ indicates density of a normal distribution.

$${y}^{′} = \frac{1}{\sqrt{2 π}} {e}^{- \frac{{( x - M )}^{2}}{2 {σ}^{2}}}$$

### Charisma (T6)

The parameter charisma T6 is determined according to a degree of symmetry of micromovements of the head and face. Maximum symmetry of movements in terms of frequency and amplitude means a high level of charisma (or attractiveness).

$$T 6 = \frac{\sum \frac{\vert {W}_{li} - {W}_{ri} \vert}{max ( {W}_{li} , {W}_{ri} )} + \sum \frac{\vert {C}_{li} - {C}_{ri} \vert}{255}}{N} \qquad [ Equation 8 ]$$

wherein,
W: Frame size of a subject frame
li: Frame size of a left column of the subject frame
ri: Frame size of a right column of the subject frame
C: Color of the subject frame.

### Energy (T7)

The energy parameter T7 is determined according to a frequency bar graph and means a standard error of vibration frequencies of the face and head and a maximum difference of vibration-frequency density (aura color size or value). As a frequency value becomes higher and a standard deviation or a degree of vibration dispersion (aura color variation) becomes lower, an energy parameter value becomes higher.

$$T 7 = \frac{M - σ}{Fps} \qquad [ Equation 9 ]$$

wherein,
M: Color size (value) of the subject's aura in a current frame (vibration-frequency density)
Fps: Frames per second
σ: Average aura color size of the subject.

### Self-Regulation (T8)

The parameter Self-regulation T8 means a total value of current positive emotions, and the Self-regulation is an average of a parameter value of balance and a parameter value of charisma.

$$T 8 = \frac{T 5 + T 6}{2} \qquad [ Equation 10 ]$$

$$C = \frac{\sum [ y ( x ) * K - {{y}^{′} ( x )}^{2}}{\sum {[ {y}^{′} ( x ) ]}^{2}}$$

wherein,
K: Normalized correlation coefficient of an initial histogram
y′: Normal-distribution density.

$$K = \frac{\sum {y}^{′} ( x )}{\sum y ( x )}$$

Wherein,
y′: General-distribution density.

$${y}^{′} = \frac{1}{\sqrt{2 π}} {e}^{- \frac{{( x - M )}^{2}}{2 {σ}^{2}}}$$

### Inhibition (T9)

The parameter Inhibition T9 means the only practical physiological value (time per second) among the entire measured psychophysiological parameters T1-T10 and means a response to a specific event (motive) for a minimum time. As a response time becomes longer, it means that inhibition is performed well.

The Inhibition T9 indicates a calculation period of an amplitude Da of a pixel brightness difference between two frames, and the Da is expressed as below.

$$Da = \frac{255}{Ca} \sum Ii \neq 0 ? 1 : 0 \qquad [ Equation 11 ]$$

wherein,
Ca: Number of pixels within a designated range
I: Brightness value of a pixel
i: Number of pixels.

### Neuroticism (T10)

The parameter neuroticism T10 means a diffusion distribution (standard deviation) of inhibition measured during a change period (silent 60 seconds). As an inhibition distribution becomes higher, it means that a psychophysiological state becomes unstable, and naturally neurotic symptoms become more pronounced. This neuroticism T10 has a value that is ten times a standard deviation of values of the inhibition T9.

Analyzing a movement trajectory of the head based on basic biological data is significantly different from human emotional analysis based on various facial micromovements presented by Paul Ekman and Fridlund. The facial micromovements show overall or temporary emotional states well, but continuous psychophysiological processes such as blood pressure, electrodermal response, or electrocardiogram used in lie detection are not measured, so effectiveness in automatic emotional analysis is low. Spatial-temporal parameters of micromovements of the head are associated with all emotional and psychophysiological state changes.

To effectively evaluate the disclosure in non-contact diagnosis of psychophysiological brain-fatigue states, psychophysiological diagnosis was performed on workers employed at a nuclear power plant. The study methods included psychological diagnostic tests such as the minnesota multiphasic personality inventory (MMPI), Cattell's sixteen personality factor questionnaire (16 PF), Raven's progressive matrices (Raven), and the luscher color test (LSC); psychophysiological evaluations related to simple and complex hand-foot reactions; physiological evaluations related to responses to moving objects; physiological heart-rate variability evaluation; and plethysmographic measurement methods, and anthropometric characteristics of the study subjects were recorded. According to the disclosure, the examination time, previously requiring an average of two and a half hours per subject, was reduced to approximately one minute.

Visualization of vibration images and normal distribution (Gaussian distribution) of frequency histograms generally correspond to emotional and physiological states. The distribution of a maximum probability density function depends on a person's energy level.

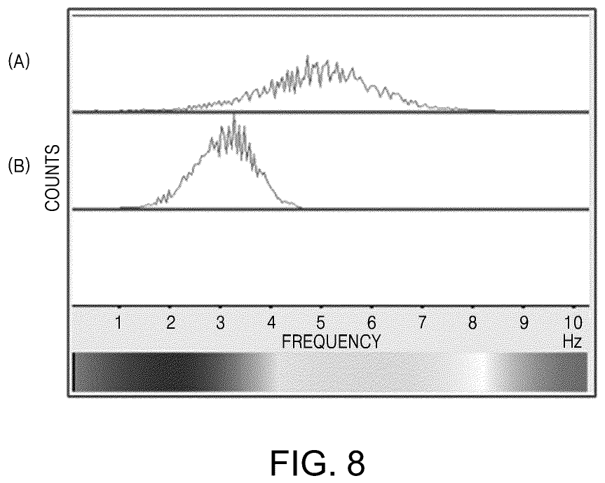

FIG. 8 illustrates a histogram of a normal psychophysiological state. As shown in FIG. 8, unlike a normal state (A), a psychophysiological state that is very fatigued with a low energy level has a maximum probability-density function shifted to the left in a frequency band.

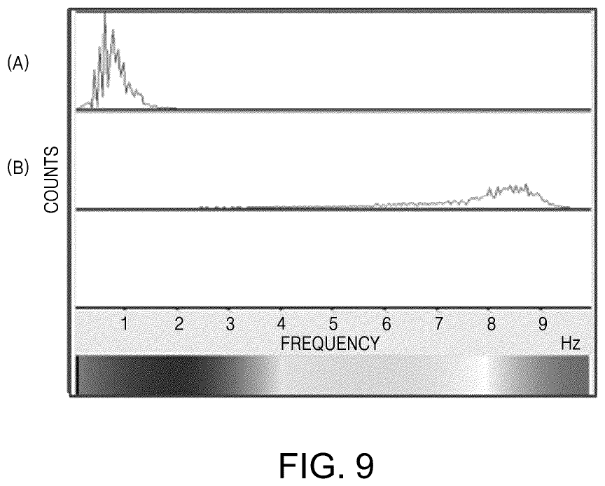

In FIG. 9, (A) shows a histogram of a very fatigued psychophysiological state, and (B) shows a histogram of a psychophysiological state of a very active person.

A psychophysiological state of a very active person has a maximum probability-density function shifted to the right in a frequency band.

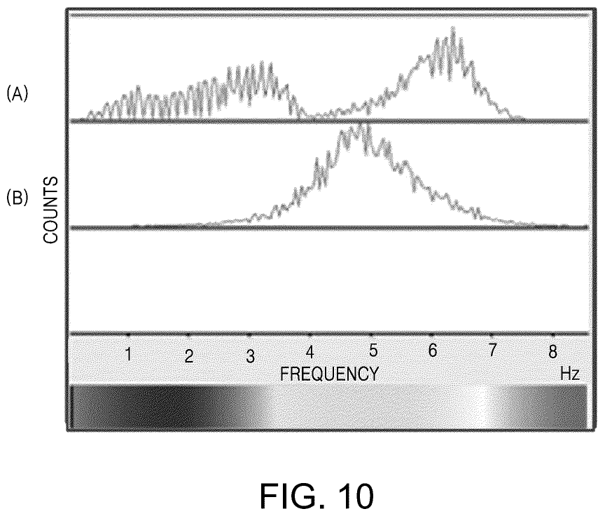

FIG. 10 illustrates frequency histograms of a worst case and a best case, wherein a frequency histogram (A) is a frequency histogram of a subject having psychopathological issues and a frequency histogram (B) is a frequency histogram of a subject in a healthy and good state.

Referring to the frequency histogram (A) of FIG. 10, obtained from a subject in a worst case, asymmetry of the frequency histogram having several peaks and a probability-density-function shape appears, which indicates emotional or physiological pathological phenomena such as aggression or stress.

A standard deviation and variance of a probability-density function depend on normal levels of emotions, and generally, smaller standard deviation and smaller variance correspond to better or positive emotional states such as a happiness level.

Spatial-temporal parameters of micromovements of the head are associated with all emotional and psychophysiological state changes.

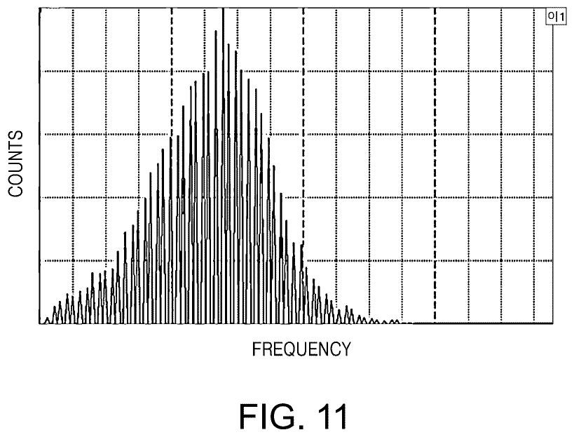

FIG. 11 illustrates a frequency histogram of a healthy and good normal state.

A method according to the disclosure, which calculates and extracts psychophysiological information PI and energy-metabolism EM parameters by using information of a mean value of a frequency histogram, a standard deviation, and a frequency having a maximum distribution, is as follows.

### 1) Extraction of Psychophysiological Information PI

A value of psychophysiological information PI is calculated as a variation ratio of a standard deviation S of a histogram to frames per second at a time point t.

A calculation formula of psychophysiological information PI(t) at a time point t is as follows.

$$PI ( t ) = ( fps ( t ) - k {( t )}^{*} S ( t ) ) / fps ( t ) \qquad [ Equation 12 ]$$

wherein,
fps(t): frames per second
k=5*sqrt (N0/100): fps-proportional constant
S(t): standard deviation of histogram.

For psychophysiological information PI actually used, a value obtained by multiplying the above-calculated value by 100 is used, and therefore this value has a range from 0 to 100.

A value of psychophysiological information PI is inversely proportional to a value of a standard deviation S of a histogram, and therefore, when the value of the standard deviation S decreases, a value of the psychophysiological information PI increases, and conversely, when the value of the standard deviation S increases, the value of the psychophysiological information PI decreases.

Accordingly, psychophysiological information PI indicates a degree of psychophysiological stability (relaxation) or instability (arousal).

(1) A large value of psychophysiological information PI means that a value of a standard deviation S of a histogram is small and that psychophysiological variability is maintained almost without change. Consequently, this indicates that the psychophysiological state is stable.

(2) Conversely, a small value of psychophysiological information PI means that a value of a standard deviation S of a histogram is large and that psychophysiological variability is changing very greatly. Ultimately, this means that the psychophysiology may be in an unstable state.

### 2) Extraction of Energy Metabolism EM

A value of energy metabolism EM is calculated as a ratio of a value of frames per second fps to the fps value fMax at which maximum frequency (vitality) of a histogram appears at a time point “t”, and a calculation formula of energy metabolism EM(t) at the time point “t” is as follows.

$$EM ( t ) = f Max ( t ) / fps ( t ) \qquad [ Equation 13 ]$$

wherein,
fps(t): frames per second
fMax(t): fps value at which maximum frequency of histogram appears.

In practice, a value of the energy metabolism EM may be obtained by multiplying the above-calculated value by 10, and therefore this value has a range from 0 to 10.

According to Equation 13 above, a value of the energy metabolism EM is proportional to the fMax value, which is the fps value at which maximum frequency of a histogram appears, and therefore, when the fMax value decreases, a value of the energy metabolism EM also decreases, and when the fMax value increases, a value of the energy metabolism EM increases. Accordingly, the energy metabolism EM indicates a degree of maximum vitality.

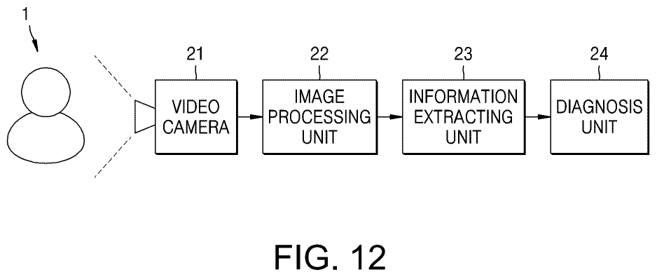

To perform psychopathological diagnosis according to an embodiment, a system illustrated in FIG. 12 may be applied.

Referring to FIG. 12, a system for psychopathological diagnosis according to the disclosure includes a video camera 21 configured to capture a subject 1, an image processing unit 22 configured to analyze an image obtained from the video camera and extract a vibration image, an information extracting unit 23 configured to extract a value of a frequency histogram and vibration parameters by using the vibration image from the image processor 22, and a psychopathological diagnosis unit 24 configured to evaluate and diagnose a psychopathological state of the subject by calculating the psychophysiological information PI and the energy metabolism EM from the frequency histogram and a plurality of parameters from the information extracting unit 23.

The extracted parameters are indicators representing emotional parameters such as aggression, stress, tension, balance, charisma, energy, inhibition, and neuroticism, as described above, and these have been described in detail above.

According to the embodiments as described above, psychopathological diagnosis of a subject may be rapidly measured in a non-contact manner. These embodiments present an innovative method that enables diagnosis of psychological and mental health conditions, including brain functional states of persons having psychopathological diseases or ordinary individuals. These embodiments may conveniently and rapidly diagnose and monitor psychophysiological states by using videos in a non-contact manner, not only in an aspect of extending existing psychophysiological diagnostic tools. Practical effects obtainable through these embodiments are as follows.

By using data obtained by capturing and analyzing videos in real time, changes in psychophysiological information and energy metabolism are exhibited, making it possible to identify and understand brain functional states and psychological emotional changes.

By providing time-series data of psychophysiological-state information and changes in a value of the energy metabolism, which may be used to identify psychopathological signs or diagnoses and severity of pathological conditions, it contributes to functional diagnosis and monitoring (management) of treatment effects. In addition, it is possible to identify whether prescribed medication for a patient causes side effects during the course of taking the medication and to determine its effectiveness.

1) Based on non-contact videos, a plurality of parameters, psychophysiological information and the value of the energy metabolism and their changes are evaluated and diagnosed simply and rapidly both remotely and on site. The system enables obtaining multidimensional relationships of psychophysiological-state characteristics, and it enables measurement not only of changes in the energy metabolism but also of changes in the psychophysiological information.

2) As a diagnostic tool for understanding psychophysiological states, the system may be used for counseling subjects, diagnosing mental health conditions of patients in hospitals, and serving as data for counseling and therapeutic activities.

3) Prevention of psychological distortion that may occur in self-report questionnaires is possible, and convenient examination is enabled even for children or individuals with developmental disabilities who have difficulty taking questionnaires, and for patients (clients) having pathological signs.

4) From a chronobiological perspective, by measuring a subject's parameters regularly and over the long term and monitoring changes in the subject's psychophysiological state, it can be expanded into a mental-health-care service.

5) By converting measured psychophysiological information into big data and applying artificial intelligence (AI) technology, it may be developed into a medical diagnostic method and device applying an AI model.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of psychopathological diagnosis, comprising:
capturing, by using a video camera, a video of a subject;
extracting, by using an image processing unit, a vibration image of the subject from the video;
extracting, from the vibration image by using an information extracting unit, changes in psychophysiological information, changes in energy metabolism, and a plurality of parameters related to emotional states of the subject; and
performing, by using a psychopathological diagnosis unit, psychopathological evaluation and diagnosis based on the changes in the psychophysiological information, the changes in the energy metabolism, and the plurality of parameters.

2. The method of claim 1, wherein the vibration image includes at least one of a vibration frequency, an amplitude, and a phase according to locations in each part of the subject.

3. The method of claim 1, wherein extracting of the changes in the psychophysiological information, the changes in the energy metabolism, and the plurality of parameters comprises extracting, by extracting frequency histograms from the vibration image, the changes in the psychophysiological information and the changes in the energy metabolism according to temporal variations.

4. The method of claim 3, wherein the psychophysiological information at a time “t” is calculated according to Equation 1 by using an average value of the frequency histograms, a standard deviation, and a maximum frequency of the frequency histograms:
$$PI ( t ) = ( fps ( t ) - k {( t )}^{*} S ( t ) ) / fps ( t ) \qquad < Equation 1 >$$
wherein,
PI(t): psychophysiological information
fps(t): frames per second
k=5*sqrt (N0/100): fps-proportional constant
S(t): standard deviation of histogram.

5. The method of claim 4, wherein the energy metabolism at a time “t” is calculated according to Equation 2:
$$EM ( t ) = f Max ( t ) / fps ( t ) \qquad < Equation 2 >$$
wherein,
EM(t): energy metabolism
fps(t): frames per second
fMax(t): fps value at which maximum frequency of histogram appears.

6. The method of claim 5, wherein the plurality of parameters include aggression, stress, tension, balance, charisma, energy, inhibition, and neuroticism, and at least two thereof are used for evaluating and diagnosing a psychopathological state.

7. The method of claim 1, wherein the plurality of parameters include aggression, stress, tension, balance, charisma, energy, inhibition, and neuroticism, and at least two thereof are used for evaluating and diagnosing a psychopathological state of the subject.

8. The method of claim 1, wherein the extracting of the changes in the psychophysiological information, the changes in the energy metabolism, and the plurality of parameters comprises extracting, by extracting frequency histograms from the vibration image, the changes of the psychophysiological information and the changes of the energy metabolism according to temporal variations, and
wherein the psychophysiological information and the energy metabolism, both at a time “t”, are calculated according to Equations 3 and 4, respectively, by using an average value of the frequency histograms, a standard deviation, and a maximum frequency of the frequency histograms:
$$PI ( t ) = ( fps ( t ) - k {( t )}^{*} S ( t ) ) / fps ( t ) \qquad < Equation 3 >$$
wherein,
PI(t): psychophysiological information
fps(t): frames per second
k=5*sqrt (N0/100): fps-proportional constant
S(t): standard deviation of histogram
$$EM ( t ) = f Max ( t ) / fps ( t ) \qquad < Equation 4 >$$
wherein,
EM(t): energy metabolism
fps(t): frames per second
fMax(t): fps value at which maximum frequency of histogram appears.

9. The method of claim 8, wherein the plurality of parameters include aggression, stress, tension, balance, charisma, energy, inhibition, and neuroticism, and at least two thereof are used for evaluating and diagnosing a psychopathological state.

10. A system for psychopathological diagnosis, the system comprising:
a video camera configured to capture a video of a subject;
an image processing unit configured to extract a vibration image of the subject from the video;
an information extracting unit configured to extract, from the vibration image, changes in the psychophysiological information and changes in the energy metabolism of the subject and a plurality of parameters related to emotional states of the subject; and
a psychopathological diagnosis unit configured to perform psychopathological evaluation and diagnosis based on the changes in the psychophysiological information, the changes in the energy metabolism, and the plurality of parameters.

11. The system of claim 10, wherein the information extracting unit is further configured to extract, by extracting frequency histograms from the vibration image, the changes of the psychophysiological information and the changes of the energy metabolism according to temporal variations.

12. The system of claim 11, wherein the information extracting unit is further configured to calculate the psychophysiological information at a time “t” according to Equation 5 by using an average value of the frequency histograms, a standard deviation, and a maximum frequency of the frequency histograms:
$$PI ( t ) = ( fps ( t ) - k {( t )}^{*} S ( t ) ) / fps ( t ) \qquad < Equation 5 >$$
wherein,
PI(t): psychophysiological information
fps(t): frames per second
k=5*sqrt (N0/100): fps-proportional constant
S(t): standard deviation of histogram.

13. The system of claim 11, wherein the energy metabolism at a time “t” is calculated according to Equation 6:
wherein,
$$EM ( t ) = f Max ( t ) / fps ( t ) \qquad < Equation 6 >$$
EM(t): energy metabolism
fps(t): frames per second
fMax(t): fps value at which maximum frequency of histogram appears.

14. The system of claim 13, wherein the information extracting unit is further configured to extract at least two of the plurality of parameters related to emotional states, including aggression, stress, tension, balance, charisma, energy, inhibition, and neuroticism.

15. The system of claim 10, wherein the information extracting unit is configured to extract at least two of the plurality of parameters related to emotional states including aggression, stress, tension, balance, charisma, energy, inhibition, and neuroticism.

16. The system of claim 10, wherein the information extracting unit is configured to:
extract, by extracting frequency histograms from the vibration image, the changes in the psychophysiological information and the changes in the energy metabolism according to temporal variations; and
calculate the psychophysiological information and the energy metabolism, both at a time “t”, according to Equations 7 and 8, respectively, by using an average value of the frequency histograms, a standard deviation, and a maximum frequency of the frequency histograms:
$$PI ( t ) = ( fps ( t ) - k {( t )}^{*} S ( t ) ) / fps ( t ) \qquad < Equation 7 >$$
wherein,
PI(t): psychophysiological information
fps(t): frames per second
k=5*sqrt (N0/100): fps-proportional constant
S(t): standard deviation of histogram
$$EM ( t ) = f Max ( t ) / fps ( t ) \qquad < Equation 8 >$$
wherein,
EM(t): energy metabolism
fps(t): frames per second
fMax(t): fps value at which maximum frequency of histogram appears.

17. The system of claim 16, wherein the information extracting unit is further configured to extract at least two of the plurality of parameters including aggression, stress, tension, balance, charisma, energy, inhibition, and neuroticism.

18. The system of claim 11, wherein the psychopathological diagnosis unit is further configured to perform psychopathological evaluation and diagnosis for the subject by applying an artificial intelligence model trained with the changes in the psychophysiological information, the changes in the energy metabolism, and at least two of the plurality of parameters selected from aggression, stress, tension, balance, charisma, energy, inhibition, and neuroticism.
